(19) United States
(12) Patent Application Publication
(10) Pub. No.: US 2023/0257183 A1
(43) Pub. Date: Aug. 17, 2023

(54) Methods For Reducing Catecholamine-Formaldehyde Adducts

(71) Applicant: Nevakar Injectables Inc., Bridgewater, NJ (US)

(72) Inventors: Reema Ajitkumar Puri, Hillsborough, NJ (US); Tushar Hingorani, Bridgewater, NJ (US); Kumaresh Soppimath, Skillman, NJ (US); Harshil H. Jain, Monmouth Junction, NJ (US)

(73) Assignee: Nevakar Injectables Inc., Bridgewater, NJ (US)

(21) Appl. No.: 18/011,664

(22) PCT Filed: Jun. 22, 2021
(86) PCT No.: PCT/US2021/038419

§ 371 Date: Dec. 20, 2022

Related U.S. Application Data
(60) Provisional application No. 63/043,372, filed on Jun. 24, 2020.

(51) Int. Cl. B65D 81/26; A61J 1/14

(52) U.S. Cl. CPC B65D 81/266; A61J 1/14; A61K 31/137

(57) ABSTRACT

Accidental formaldehyde adduct formation of catecholamines in a catecholamine-containing formulation in a polymeric container is significantly reduced where a metal-based compound is used as an oxygen scavenger located between the polymeric container and a compromised secondary container that encloses the polymeric container.

This application claims the benefit of U.S. Provisional Application No. 63/043,372, filed Jun. 24, 2020, the disclosure of which is hereby incorporated by reference in its entirety.

### FIELD OF THE INVENTION

The field of the invention is preservation of various pharmaceutical compositions, and particularly prevention of formaldehyde-condensation products of catecholamines in pharmaceuticals upon accidental oxygen ingress.

### BACKGROUND OF THE INVENTION

The background description includes information that may be useful in understanding the present invention. It is not an admission that any of the information provided herein is prior art or relevant to the presently claimed invention, or that any publication specifically or implicitly referenced is prior art.

All publications and patent applications herein are incorporated by reference to the same extent as if each individual publication or patent application were specifically and individually indicated to be incorporated by reference. Where a definition or use of a term in an incorporated reference is inconsistent or contrary to the definition of that term provided herein, the definition of that term provided herein applies and the definition of that term in the reference does not apply.

Epinephrine is a sympathomimetic catecholamine (adrenergic agent) designated chemically as 4-[1-hydroxy-2(methylamino)ethyl]-1,2-benzenediol, a white, microcrystalline powder and has the following structural formula:

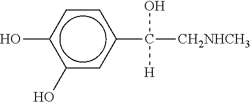

Most commonly, epinephrine is marketed as a concentrated form for injection (1 mg/mL), which must be diluted with a proper diluent such as dextrose or dextrose/NaCl. Unfortunately, all or almost all of the known diluted epinephrine formulations that are commercially available lack storage stability. Indeed generally, diluted epinephrine solutions must be discarded within one day after reconstitution when stored at room temperature. Since diluted epinephrine solutions are unstable with limited shelf life, such solutions are almost never sold or shipped, but must be prepared at hospitals or doctor's offices for immediate use. More recently, diluted and ready-to-administer formulations have been developed as described in U.S. Pat. No. 10,653,646, where a specific formulation at pH of between 3.0-4.7 included a metal ion chelator and that provided desirable long-term stability, even at very low concentrations.

Epinephrine can be subject to multiple degradation pathways. For example, exposure to air or light (typically via auto-oxidation) often results in the formation of adrenochrome and melanin. The rate of this reaction increases with increased pH, increased temperature, and in the presence of metal ions such as aluminum from various rubbers and iron from amber glass ware. Epinephrine solutions may also lose potency as a result of racemization from the biologically active R-isomer to the biologically inactive S-isomer. Such racemization notably increases with increasing temperatures. Epinephrine is also rapidly destroyed in alkaline solutions by aldehydes, weak oxidizing agents, and atmospheric oxygen.

To reduce oxidative degradation, epinephrine formulations may include one or more antioxidants such as sodium metabisulfite or sodium bisulfite to protect catecholamines or adrenergic compounds against auto-oxidation. For example, where epinephrine and sodium metabisulfite were used at a ratio of about 1:0.005 to about 1:15 by weight, formulations with desirable storage stability were obtained. However, such antioxidants have been associated in at least some cases with severe allergic reactions. In addition, sodium bisulfite can react directly with epinephrine in aqueous solution upon exposure to room light, probably due to the conversion of superoxide (O₂⁻) radicals to highly reactive hydroxyl (·OH) radicals by bisulfite (see e.g., Photo destabilization of Epinephrine by sodium metabisulfite (PDA J Pharm Sci Technol. 2000 March-April; 54(2):136-43). In addition, the potency of epinephrine could be further substantially degraded during shelf life storage due to radical-mediated reactions. Therefore, in view of the reactivity of epinephrine with widely used antioxidants, use of such compounds is not advisable in epinephrine containing formulations.

In yet other attempts to increase storage stability of epinephrine formulations, inclusion complexes of epinephrine with native or modified cyclodextrin derivatives have been prepared as is described in US 2018/0028671. While such compositions reduced thermal and/or oxidative degradation, the use of complexing agents rendered manufacture more difficult. Moreover, the bioavailability of the epinephrine may be reduced at least in some formulations. Furthermore, when epinephrine is diluted with water, such water often contains dissolved oxygen, as does the head space in the container. The oxygen in the water acts to degrade the epinephrine or other oxygen sensitive drug, increasing degradants and decreasing shelf life.

To protect epinephrine from atmospheric oxygen and light, epinephrine formulations, and particularly low-concentration ready-to-administer epinephrine formulations, can be packaged with a metallized overwrap that forms a strong barrier for light and oxygen ingress. Where desired, an oxygen scavenger (non-metal containing and metal-containing) may be placed between the metallized overwrap and the container that contains the epinephrine as taught in US 2018/0028671 and WO 2020/057134. Advantageously, such scavenger will eliminate atmospheric oxygen in the space between the overwrap and the epinephrine container and will maintain or even reduce dissolved oxygen in the epinephrine container. However, when the overwrap is compromised (e.g., punctured or improperly sealed), various degradation products may once more arise.

Thus, even though various systems and methods of preserving epinephrine formulations are known in the art, all or almost all of them suffer from several drawbacks. Therefore, there remains a need for compositions and methods for improved compositions and methods to preserve epinephrine compositions, and especially ready-to-administer epinephrine formulations.

### SUMMARY OF THE INVENTION

The inventive subject matter is directed to various compositions and methods of preserving epinephrine and other catecholamine compositions, and especially prevention of formaldehyde adduct formation in packaged ready-to-administer formulations where the package is compromised and subject to inadvertent oxygen ingress.

In one aspect of the inventive subject matter, the inventors contemplate a method of protecting a catecholamine in a catecholamine-containing formulation from accidental formaldehyde adduct formation that includes a step of placing a polymeric container into an overwrap, wherein the polymeric container encloses the catecholamine-containing formulation; and a further step of placing a metal-containing oxygen scavenger in a space between the polymeric container and the overwrap, and sealing the overwrap. Most typically, the accidental formaldehyde adduct formation is triggered by improper sealing or puncture of the overwrap.

In some embodiments, the catecholamine is epinephrine or norepinephrine, and the polymeric container is manufactured from a polyolefin selected from the group consisting of polyvinyl chloride, polyethylene, polypropylene, polyethylene terephthalate, an ethylene-propylene copolymer, ethylene vinyl acetate, or a copolyester ether polymer. Most typically, but not necessarily, the polymeric bag has a volume of between 100 mL and 1000 mL, and the overwrap is a metalized polymer overwrap.

In further embodiments, the metal-containing oxygen scavenger is a StabilOx scavenger, an SS100 scavenger, or a ZPT scavenger, and/or the puncture has an area of less than 5 mm². In still further embodiments, less than 1% of formaldehyde adduct are formed at 25° C. within 1 month following break or puncture.

Therefore, the inventors also contemplate a packaged pharmaceutical composition containing a catecholamine in a polymeric container, wherein the polymeric container is contained in an overwrap. Such packaged composition will preferably comprise a metal-containing oxygen scavenger in a space between the polymeric container and the overwrap, wherein the oxygen scavenger is present in an amount that prevents accidental formaldehyde adduct formation upon a break or puncture of the overwrap.

Viewed from another perspective, the inventors also contemplate a method of reducing formaldehyde adduct formation of a catecholamine (e.g., epinephrine or norepinephrine) in a closed environment subject to oxygen ingress that includes a step of scavenging oxygen in the closed environment using a metal-containing oxygen scavenger, wherein the metal-containing oxygen scavenger is located in the closed environment and is not in direct contact with the catecholamine.

Preferably, the metal-containing oxygen scavenger is a StabilOx scavenger, an SS100 scavenger, or a ZPT scavenger or similar product, and the closed environment is a closed space between a container that contains the catecholamine and an overwrap enclosing the container. Most typically, the metal-containing oxygen scavenger is present in an amount that leads to equal or less than 1% of formaldehyde adduct at 25° C. within 1 month.

Various objects, features, aspects and advantages of the inventive subject matter will become more apparent from the following detailed description of preferred embodiments, along with the accompanying drawing figures in which like numerals represent like components.

### DETAILED DESCRIPTION

The inventors have discovered that accidental low-grade oxygen ingress into the space between a container containing a catecholamine-containing formulation, such as an epinephrine formulation, (and especially a ready-to-administer epinephrine formulation) and an overwrap enclosing the container leads to the formation of various aldehyde adducts, and particularly formaldehyde and/or acetaldehyde adducts of epinephrine in the epinephrine formulation. Therefore, even formulations that would otherwise be storage stable can accumulate degradation products over time where the overwrap is compromised. Such is especially disadvantageous where the damage to the overwrap is not readily visible as may be the case with an improper seal or a pinhole defect. In this context, it should be noted that the term “aldehyde adduct” expressly includes condensation product with an aldehyde, and particularly with formaldehyde.

In most cases, overwraps are used to protect a container and its contents from various environmental conditions. For example, overwraps can be colored or metallized to provide a light barrier for light-sensitive agents. In other examples, overwraps will also often form a strong barrier against liquids and gases. Thus, many overwraps will have low to very low oxygen transmission rates, and almost always have an oxygen transmission rate that is significantly lower than that of the container that is enclosed by the overwrap. Due to this fact, oxygen scavengers will not only effectively reduce oxygen concentration in the space between the overwrap and the container, but also lower dissolved oxygen concentrations in a liquid that is held in the container as the oxygen flux is predominantly from the liquid pharmaceutical formulation into the space between the two containers, and significantly less from outside the overwrap into the space between the two containers. Viewed from a different perspective, in devices, system, and methods of the inventive subject matter, the oxygen diffusion rate of the inner bag will be greater than the oxygen diffusion rate of the outer bag.

The inventors have now unexpectedly discovered that the type of oxygen scavenging material that is placed in the space between a drug container and an overwrap will have a significant impact on formaldehyde adduct formation where the drug is a catecholamine such as epinephrine or norepinephrine. Notably, where non-metal-based oxygen scavengers were used, formaldehyde/acetaldehyde adduct formation with the catecholamine was observed while chemical storage stability was otherwise not significantly changed. On the other hand, where metal-containing oxygen scavengers were used, formaldehyde/acetaldehyde adduct formation with the catecholamine was substantially less, or even not detectable. In this context, it should be appreciated that metal-containing scavengers are typically discouraged with catecholamine-type drugs, as metal catalyzed reactions present a major degradation pathway for catecholamines.

In view of the foregoing, a method of protecting a catecholamine in a catecholamine-containing formulation from inadvertent formaldehyde condensation product formation is provided. The method comprises placing a polymeric container enclosing the catecholamine-containing formulation into the overwrap, placing a metal-containing oxygen scavenger in a space between the polymeric container and the overwrap, and sealing the overwrap from the environment to form a sealed overwrap. The inadvertent formaldehyde condensation product formation is triggered by improper sealing or puncture of the overwrap.

In other embodiments, another method of protecting the catecholamine in the catecholamine-containing formulation from formaldehyde condensation product formation is provided. The method comprises placing the polymeric container containing the catecholamine into the overwrap, placing the metal-containing oxygen scavenger in the space between the polymeric container and the overwrap, and partially, but not completely, sealing the overwrap from the environment to form a partially sealed overwrap. The phrase “partially, but not completely” with reference to sealing the overwrap means that the space between the polymeric container and the overwrap remains in fluid communication with the environment, typically, through an inadvertent opening (such as incomplete seal or small break at the seal) or puncture in the overwrap.

A kit for use in protecting the catecholamine in the catecholamine-containing formulation from inadvertent formaldehyde condensation product formation is also provided. Such kits comprise each of the components assembled together, such as the overwrap, the polymeric container comprising the catecholamine with the polymeric container contained in the overwrap, and the metal-containing oxygen scavenger in the space between the polymeric container and the overwrap. The metal-containing oxygen scavenger is present in an amount that prevents inadvertent formaldehyde adduct formation upon a break or puncture of the overwrap. The kit may comprise one or more additional components such as additional devices, desirable from a commercial and user standpoint for the pharmaceutical packaging systems. Non-limiting examples of such materials include, but not limited to, buffers, diluents, filters, needles, syringes; adaptors, waste receptacles, and/or labels listing contents and/or instructions for use, and package inserts with instructions for use associated with the pharmaceutical packaging system. A set of instructions will also typically be included.

The formaldehyde condensation product formation is influenced by the presence of oxygen via low grade oxygen ingress into the space from the environment. Most typically, the low-grade oxygen ingress will be higher than the oxygen transmission rate (OTR) of the overwrap, which may for various metal coated overwraps be between 0.01 and 0.1, or between 0.05-0.5, or between 0.1 and 1.0, or between 0.5 and 5, or between 1.0 and 10.0. Where the overwrap is not coated with a metal film, the OTR may be between 0.01 and 0.01, or more commonly between 1.0 and 10, and in less preferred cases between 100 and 500, in accordance with ASTM D3985. Viewed form a different perspective, the low-grade oxygen ingress may be at least 2-fold, or at least 3-fold, or at least 5-fold, or at least 10-fold, or at least 30-fold, or at least 50-fold, or at least 100-fold of the OTR of the overwrap. Therefore, and viewed form a different perspective, the low-grade oxygen ingress may have an OTR of between 1-10, or between 10-50, or between 50-250, or between 250-1,000, or between 1,000-5,000, or between 5,000-10,000 and in some cases even higher.

In various embodiments, the presence of oxygen results from partial sealing of the overwrap. In other embodiments, the presence of oxygen results from a defect in the overwrap. The term “influenced” with reference to the presence of oxygen means that oxygen is a direct or indirect contributing factor to the formaldehyde condensation product formation but may not be the only factor. In various embodiments, oxygen from the environment moves into the space through an opening in the overwrap resulting from partial sealing of the overwrap. In other embodiments, oxygen from the environment moves into the space through an opening in the overwrap resulting from a defect in the overwrap. It is to be appreciated that the OTR of the overwrap may relate to the movement of oxygen through the opening resulting from partial sealing or defect in the overwrap.

Most typically, such adduct formation (e.g., formaldehyde condensation product) was noted where the oxygen ingress into the space between the overwrap and the container was slow, and in most cases due to a pinhole defect or an improper or compromised seal that was formed when closing the overwrap. For example, a typical pinhole defect will have an open area of between 0.01-0.05 mm², or between 0.05-0.1 mm², or between 0.1-0.3 mm², or between 0.2-0.6 mm², or between 0.6-0.9 mm², or between 0.9-1.2 mm², or between 1.2-1.7 mm², or between 1.7-2.5 mm², or even larger. Likewise, seal defects in the overwrap may extend between 0.01-0.1 mm, or between 0.1-0.5 mm, or between 0.5-1.0 mm, or between 1.0-3.0 mm, or even longer. Of course, multiple defects are also expressly contemplated herein.

Most typically, the overwrap will be a multi-layer polymeric material that may also include a metal or metallized layer. For example, suitable overwraps may include multiple layers with at least one layer comprising polyvinyl chloride, polyethylene, polypropylene, polyethylene terephthalate, various co-polymers such as ethylene-propylene copolymers, ethylene vinyl acetate, co-polyester ether polymers, nylon, or combinations thereof, while suitable metal layers especially include aluminum and copper. For example, contemplated containers may be packaged in an aluminized foil pouch or single- or multi-layer overwrap with an oxygen scavenger (secondary packaging), or aluminum pouches containing an oxygen absorbing layer (secondary packaging), where the outer pouch or packaging, that does not exhibit low grade oxygen ingress into the space from the environment, has an oxygen transmission rate (OTR) between 0.0005 to 0.005, or between 0.005 to 0.05, or between 0.05 to 0.5, or between 0.5 to 2.0, or between 1.0 to 5.0 cc/100 int/24 hrs. Among other examples, a suitable overwrap may comprise a polypropylene base layer that is coupled to a thin aluminum layer (e.g., thickness between 10 and 50 micrometer), which may be covered by an oriented polyester layer, or a PET layer, an aluminum foil layer, a nylon layer, and/or a polypropylene layer (e.g., commercially available as PAKVF4PCA; IMPAK CORPORATION or MEDIFLEX AUAT™ from Amcor Flexibles, Gent, Belgium).

Containers for the pharmaceutical formulations may vary considerably and suitable containers include polymeric bottles, syringes, and flexible bags. Most typically, such containers will be manufactured from polypropylene, PET, and/or various polyesters, and the skilled artisan will be readily able to select a proper material for a given pharmaceutical formulation.

An advantageous feature of the metal-containing oxygen scavenger in the methods and kits described herein is the absorbance and removal of oxygen present in the overwrap to prevent inadvertent formaldehyde adduct formation upon a break or puncture of the overwrap. With respect to metal-containing oxygen scavenger, it is contemplated that all scavengers are deemed suitable so long as they include one or more metals as the component that react with oxygen, and most typically contemplated oxygen scavengers will include iron as a metal component. As will be readily appreciated, such metal(s) may be further combined with a salt (e.g., NaCl, KCl, etc.) and/or hygroscopic agent to promote oxidation of the metal. Metal-containing oxygen scavengers include elemental iron as well as iron oxide, iron hydroxide, iron carbide and the like. Other metals for use as oxygen absorbers include nickel, tin, copper and zinc. Metal-based oxygen absorbers are typically in the form of a powder to increase surface area. Powder formation of the metal-containing oxygen scavenger is by any known method including, but not limited to, atomization, milling, pulverization, and electrolysis. The metal-containing oxygen scavenger are contemplated to be in any size or shape including sachet, pouch, capsule, label, strip, patch, canister, cartridge, lining, sticker, etc. that is placed inside of the overwrap as well as part of the overwrap itself.

Where desired, additional components may be included to further absorb various agents, and especially volatile organic components. Non-limiting examples of suitable additional components include low molecular weight organic compounds such as ascorbic acid, sodium ascorbate, catechol and phenol, activated carbon and polymeric materials incorporating a resin and a catalyst. There are numerous metal-containing oxygen scavengers commercially available, and all of those are deemed suitable for use herein. For example, suitable metal-containing oxygen scavengers include Ageless Oxygen Absorber from Mitsubishi Chemicals, Oxyguard from Uline, O-busters from AGM, etc. On the other hand, less preferred non-metal-based oxygen scavengers may include various antioxidants such as ascorbic acid, polyphenols, carbohydrazide, tannin, and/or sulfite, or combinations of ethylenically unsaturated hydrocarbons with a transition metal that catalyze an oxidation reaction of the double bond. In exemplary embodiments, the metal-containing oxygen scavenger comprises an iron-containing oxygen scavenger. Non-limiting examples are commercially available under the trade names StabilOx, SS100, and ZPT.

In some preferred aspects, the epinephrine composition is a ready-to-administer formulation wherein epinephrine is present at a concentration of between about 0.001 to about 0.07 mg/ml, or between about 0.005 to about 0.07 mg/ml, or between about 0.005 to about 0.05 mg/ml, or between about 0.005 to about 0.03 mg/ml, or between about 0.005 to about 0.02 mg/ml. Viewed from a different perspective, while higher concentration epinephrine compositions are also contemplated, some exemplary formulations will include epinephrine at a concentration of equal or less than about 0.07 mg/ml, equal or less than about 0.05 mg/ml, or even equal or less than about 0.02 mg/ml, such that no dilution is required prior to injection. Therefore, epinephrine may be present in the ready-to-administer formulation at a concentration of between about 0.005 mg/ml and about 0.050 mg/ml, or at a concentration of between about 0.006 and about 0.010 mg/ml, or at a concentration of between about 0.010 and about 0.025 mg/ml, or at a concentration of between about 0.025 and about 0.045 mg/ml. Exemplary suitable formulations are disclosed in U.S. Ser. No. 17/154,516, U.S. Pat. Nos. 10,159,657, and 10,653,646, all incorporated by reference herein.

Of course, it should be appreciated that epinephrine may be present in the composition as base or in form of a pharmaceutically acceptable salt, including a hydrochloride salt, a bitartrate salt, and a borate salt as such salt forms substantially increase the solubility of epinephrine in an aqueous medium. Moreover, it is generally preferred that substantially all of the epinephrine is in the R-isomer form (e.g., at least about 85 mol %, or at least about 90 mol %, or at least about 95 mol %, or at least about 97 mol %, or at least about 99 mol %). In still further preferred aspects, the aqueous pharmaceutically acceptable carrier is water, which may further include one or more polar and/or protic solvents that will typically form a single-phase solvent system with the water.

With further respect to the aqueous pharmaceutically acceptable carrier, the carrier and/or the ready-to-administer epinephrine composition may have a dissolved oxygen content of equal or less than about 5.0 ppm or equal or less than about 3.0 ppm or equal or less than about 2.5 ppm or equal or less than about 2.0 ppm or equal or less than about 1.5 ppm or equal or less than about 1.0 ppm 0, (typically during compounding and/or after 1 month of storage at 25° C.+/−2° C.). To that end, the carrier and/or ready-to-administer epinephrine composition can be subjected to sparging with an inert gas (e.g., argon, helium, freons, nitrogen, etc.), vacuum stripping under agitation, storage under an inert gas headspace, storage in a polymeric container with metallized over-container that further includes the metal-containing oxygen scavenger.

In other aspects of the inventive subject matter, contemplated ready-to-administer epinephrine composition can have a mildly acidic pH of between about 3.0 and about 5.5 or between about 4.0 and about 6.0, such as a pH of between about 3.5 and about 4.5 or a pH of between about 3.8 and about 4.2. Most typically the pH of the ready-to-administer epinephrine composition will be less than about 5.0 and more typically less than about 4.5, and most typically less than about 4.3, but higher than about 3.0, more typically higher than about 3.5, and most typically higher than about 3.7. As will be readily appreciated, the pH can be adjusted using various acids (e.g., HCl) and bases (e.g., NaOH). Moreover, and where desired, the pH can be stabilized using a buffer, typically at a relatively low strength. For example, suitable buffers may be present at a concentration of between about 1 mM and about 50 mM, or between about 10 mM and about 25 mM, or between about 20 mM and about 40 mM. Among other choices, contemplated buffers include organic and inorganic buffers, as well as amphoteric buffers. For example, suitable buffers include an acetate buffer, a citrate buffer, a phosphate buffer, a tartrate buffer, and a borate buffer, which may be adjusted to a pH in the range of between about 3.7 and about 4.3, or between about 3.7 and about 4.0, or between about 3.8 and about 4.1, or between about 3.9 and about 4.2, or between about 4.0 and about 4.2. Notably, such pH ranges provided remarkable stability for low concentrations of epinephrine, especially where epinephrine was combined with a chelator and optional tonicity agents.

Moreover, in further contemplated aspects, the ready-to-administer epinephrine composition will also include one or more chelating agents, and particularly metal ion chelators to slow down the baseline and metal ion-stimulated autoxidation of epinephrine. For example, suitable chelators include various bicarboxylic acids, tricarboxylic acids, and aminopolycarboxylic acids such as ethylenediaminetetraacetic acid (EDTA), ethylene glycol-bis(β-aminoethyl ether)-N,N,N′,N′-tetraacetic acid (EGTA), and penta(carboxymethyl)di-ethylenetriamine (DTPA), and salts and hydrates thereof. While not limiting to the inventive subject matter, it is contemplated that the metal ion chelators will slow down both the baseline and metal ion-stimulated autoxidation of epinephrine. Notably, the inventors unexpectedly observed that the desirable effect of the chelators was observable at relatively low concentrations of the chelators. For example, reduction of the baseline and metal ion-stimulated autoxidation of epinephrine was observed at chelator concentrations of between about 1 μg/ml and about 10 μg/ml, and between about 10 μg/ml and about 100 μg/ml. Thus, preferred chelator concentrations will be between about 1 μg/ml and about 50 μg/ml, or between about 5 μg/ml and about 25 μg/ml. Interestingly, the chelators, and especially the aminopolycarboxylic acids retained stabilizing effect despite the relatively low pH favoring protonated forms of the chelators.

With respect to suitable tonicity agents, pharmaceutically acceptable salts are generally preferred to adjust/increase tonicity. For example, NaCl may be employed at a concentration of at least about 0.6 wt %, or at least about 0.7 wt %, or at least about 0.8 wt %, or at least about 0.9 wt %. Thus, suitable salt concentrations will typically be between about 0.6 wt % and about 1.2 wt %. Depending on the particular salt concentration, additional tonicity agents may be added and such suitable agents include glycerol, thioglycerol, mannitol, lactose, and dextrose. The amount of tonicity adjusting agent used can be adjusted to obtain osmolality of the formulations, typically in the range of about 260 to about 340 mOsm/kg. An osmometer can be used to check and adjust the amount of tonicity adjusting agent to be added to obtain the desired osmolality.

It should further be appreciated that contemplated compositions are substantially free of antioxidants (i.e., do not include antioxidants in an amount effective to reduce degradation of total epinephrine by at least about 1% when stored over a period of at least three months at 25° C.+/−2° C.). Therefore, and viewed from a different perspective, antioxidant-free and storage stable ready-to-administer epinephrine composition will include antioxidants in an amount of equal or less than about 0.01 wt %, or equal or less than about 0.005 wt %, or equal or less than about 0.001 wt %, or equal or less than about 0.0005 wt %, or equal or less than about 0.0001 wt %. Remarkably, despite the lack of antioxidants added to the formulation, the ready-to-administer epinephrine composition had unexpected storage stability over extended periods with regard to both oxidation/degradation and isomerization. For example, in some embodiments, the ready-to-administer epinephrine composition has, after storage of at least one month at 25° C.+/−2° C., total impurities of equal or less than about 0.7% and equal or less than about 2% S-isomer content. Moreover, and as is shown in more detail below, such storage stability extended to at least 3 months, at least 6 months, at least 9 months, at least 12 months, at least 15 months, or at least 18 months (when stored at a temperature of between about 2 and about 40° C.).

For example, certain ready-to-administer epinephrine composition had, after storage of at least one month at not less than 25° C., total impurities of equal or less than about 0.5% and equal or less than about 1% S-isomer content, or had, after storage of at least one month at 25° C.+/−2° C., total impurities of equal or less than about 0.2% and equal or less than about 1.5% S-isomer content, or had, after storage of at least one month at 25° C.+/−2° C., total impurities of equal or less than about 0.5% and equal or less than about 1% S-isomer content, or had, after storage of at least one month at 25° C.+/−2° C., total impurities of equal or less than about 0.3% and equal or less than about 0.7% S-isomer content, or had, after autoclaving, total impurities of equal or less than about 0.5% and equal or less than about 2.0% S-isomer content, or had, after autoclaving, total impurities of equal or less than about 0.2% and equal or less than about 1.5% S-isomer content.

With respect to the sterilization of contemplated formulations it should be appreciated that contemplated formulations may be sterilized using all known manners of sterilization, including filtration through 0.22 micron filters, heat sterilization, autoclaving, radiation (e.g., gamma, electron beam, microwave). Unexpectedly, and as shown in more detail below, the inventors have also discovered that contemplated formulations were heat stable and did not undergo significant isomerization, even under conditions of sterilization (exposure to high-pressure saturated steam) at 121° C. for at least 5, or at least 10, or at least 15 minutes. Thus, terminal sterilization to sterility is possible using contemplated compositions and methods.

The formulations contemplated herein can also be filtered through a 0.22 micron filter, and filled in to a polyethylene, polypropylene or low-density polyethylene containers in a blow-fill-seal (BFS) process. BFS is a form of advanced aseptic manufacturing wherein the container is formed, filled, and sealed in one continuous, automated system not requiring human intervention. The process begins with the extrusion of plastic granules in the form of a hot hollow pipe of molten plastic called a parison. The next step is the blow molding of the container with an open top through which the container is filled, all while the plastic remains hot and in a molten state. Once filled, the container is hermetically sealed and cooled. The blow-fill seal process can take several seconds, and contemplated ready-to-administer compositions advantageously are formulated to withstand the temperature and pressure requirements without substantial degradation of epinephrine (e.g., less than about 5 wt %, less than about 3 wt %, less than about 2 wt %, less than about 1 wt % degradation).

Once the epinephrine formulations are filled in large volume polymeric, semi-permeable infusion containers (e.g., BFS container or flexible IV bags), the containers can optionally be layered or covered with a secondary packaging system including an aluminum pouch or other oxygen scavenger. For example, the BFS containers can further be sealed in an oxygen and moisture barrier blister packaging. The blister packaging can comprise one or more layers, and the one or more layers can include aluminum foil or other oxygen absorber having an oxygen transmission rate (OTR) as discussed above. Additionally or alternatively, one or more oxygen absorbers (metal or metal free, organic material) can be incorporated into any portion of the BFS container, the secondary packaging system, or between the two (e.g., between the BFS container and the multi-layer packaging) such that the oxygen absorber removes at least a portion of oxygen from the air surrounding said oxygen-sensitive drug (e.g., dissolved oxygen in composition and/or oxygen in any headspace). A beneficial feature of the oxygen absorber is the absorbance and removal of oxygen present in the primary packaging and in the liquid drug itself. Notably, it was found that the oxygen absorber also removed residual headspace oxygen in the primary packaging and also dissolved oxygen in the liquid over time, thereby further improving stability of epinephrine.

For example, the polymeric container may be configured as a flexible bag with a volume of at least 100 ml, or at least 200 ml, or at least 300 ml, or at least 400 ml, or at least 500 ml, or at least 1,000 ml, wherein the polymeric bag may be manufactured from polyvinyl chloride, polyethylene, polypropylene, polyethylene terephthalate, various co-polymers such as ethylene-propylene copolymers, ethylene vinyl acetate, co-polyester ether polymers, etc. Such polymeric containers may be, but not necessarily have a reduced oxygen permeability. As described above, these containers or bags may then be packaged into an overwrap (or other secondary package) that has one or more additional properties that help maintain stability. For example, additional properties include light-absorbing or non-transparent films to block or at least significantly reduce ingress of light of a wavelength and/or energy sufficient to initiate photolytic degradation. Other additional properties include reduced oxygen permeability that can be achieved in a variety of manners, including multi-layered polymer and/or metal films that may also include oxygen scavenging materials. For example, a suitable overwrap may comprise a polypropylene base layer that is coupled to a thin aluminum layer (e.g., thickness between 10 and 50 micrometer), which may be covered by an oriented polyester layer (e.g., commercially available as MEDIFLEX AUAT™ from Amcor Flexibles, Gent, Belgium).

The commercially available and concentrated formulation, Epinephrine Injection USP (1 mg/mL) will after dilution in dextrose or dextrose and sodium chloride injections quickly develop colored impurities. In contrast, and as is shown in more detail below, the antioxidant-free and storage stable ready-to-administer epinephrine compositions presented herein are stable over extended periods at room temperature as well as under refrigeration (e.g., temperature between about 2 and about 8° C.). Impurities resulting from chemical reactions in solution remain within acceptable limits (e.g., less than about 10 wt %, less than about 5 wt %, less than about 4 wt %, less than about 3 wt %, less than about 2 wt %, less than about 1 wt %) over long term storage (e.g., at least 1 month, at least 2 months, at least 3 months, at least 6 months, at least 9 months, at least 12 months, at least 18 months). Therefore, epinephrine formulations of the inventive subject matter can be provided in a ready-to-administer form to avoid the inconvenience associated with diluting a concentrated small volume epinephrine parenteral formulation into infusion diluents prior to infusion. The ready-to-administer formulations also eliminate microbial contamination risks and calculation errors associated with dilution. In certain aspects of the inventive subject matter, contemplated formulations will be available in a range of concentrations commonly required by medical practitioners for emergency restoration of blood pressure in cases of acute hypotension. Thus, ready-to-administer formulations are formulations that can be administered to a patient in need thereof without prior dilution (typically at the point of care such as a hospital or physician office) from a previously stored solution having a higher epinephrine concentration. Viewed from a different perspective, the ready-to-administer formulations presented herein can be received and stored at a care facility, and then directly used for administration without prior dilution.

Therefore, inventors contemplate various kits, compositions, and methods in which a container that contains a catecholamine formulation (e.g., epinephrine or norepinephrine) is packaged into an overwrap that has an oxygen transmission rate that is lower than the oxygen transmission rate of the container. A metal-based oxygen scavenger is then placed between the container and the overwrap and the overwrap is sealed to form a hermetically closed pouch (optionally after evacuating the space or flushing the space with a gas having less than 20% 02 such as chemically pure argon or nitrogen gas). Such packaging will protect the catecholamine in the container from forming an aldehyde adduct (e.g., especially a formaldehyde or acetaldehyde adduct) when the overwrap is compromised by a pinhole, puncture, tear, or improper seal to close the overwrap.

### Examples

For determination of formaldehyde and acetaldehyde, and unless noted otherwise, the inventors performed HPLC analysis using protocols well known in the art. More specifically, quantitative determination of formaldehyde and acetaldehyde was performed by derivatizing formaldehyde and acetaldehyde with DNPH reagent. The Formaldehyde-DNPH and Acetaldehyde DNPH complex was analyzed using isocratic HPLC method with UV detection. A HPLC column with a C-8 stationary phase was used for chromatographic analysis. The mobile phase was prepared by mixing Acetonitrile and water. Quantitation of formaldehyde and acetaldehyde was accomplished by comparing corresponding peak areas from a sample solution chromatogram to that of the external reference standard solution of known concentration.

To identify potential sources for reactive aldehydes, and particularly formaldehyde and acetaldehyde, the inventors determined the formaldehyde and acetaldehyde content of various reagents used in the preparation of the epinephrine compositions. Exemplary results for free formaldehyde and acetaldehyde are provided in Table A below. As can be seen from the data, none of the reagents provided a significant source of formaldehyde and acetaldehyde, with dextrose being a minor contributor of reactive aldehyde formation.

TABLE A

| | | | Formaldehyde | Acetaldehyde |
|---|---|---|---|---|
| | Formaldehyde | Acetaldehyde | concentration | concentration) |
| | concentration | concentration | (Corrected for | (Corrected for |
| Sample | (ng/mL) | (ng/mL) | water)(ng/mL) | water) (ng/mL) |
| Sample 1 T0 Milliq water | 6.982 | — | 0.000 | — |
| Sample 2 T0 NaCl in water | 7.270 | — | 0.288 | — |
| Sample 3 T0 EDTA in water | 7.415 | — | 0.433 | — |
| Sample 4 T0 Tartaric acid in water | 6.796 | — | −0.186 | — |
| Sample 5 T0 HCl in water | 7.596 | — | 0.614 | — |
| Sample 6 T0 NaOH in water | 6.370 | — | −0.612 | — |
| Sample 7 T0 0.9% Sodium chloride | 29.497 | 29.202 | 22.515 | 29.202 |
| injection B-Braun(bag2) | | | | |
| Sample 8 T0 0.9% sodium | 15.777 | 26.241 | 8.795 | 26.241 |
| chloride B-Braun(bag 1) | | | | |
| Sample 9 T0 5% dextrose | 226.858 | 342.549 | 219.876 | 342.549 |
| injection Baxter (bag 2) | | | | |
| Sample 10 T0 5% dextrose | 238.730 | 406.938 | 231.748 | 406.938 |
| injection Baxter (bag 1) | | | | |
| Sample 11 T0 Milliq water in | 5.446 | 23.199 | −1.536 | 23.199 |
| Technoflex bag (bag 2)(autoclaved) | | | | |
| Sample 12 T0 Milliq water in | 6.273 | 26.570 | −0.709 | 26.570 |
| Technoflex bag (bag 1)(autoclaved) | | | | |
| Sample 13 T0 Milliq water in | 3.860 | 27.680 | −3.122 | 27.680 |
| Galenica bag (bag 2)(autoclaved) | | | | |
| Sample 14 T0 Milliq water in | 4.188 | 25.825 | −2.794 | 25.825 |
| Galenica bag (bag 1)(autoclaved) | | | | |

Pinhole Study I (short-term study): The inventors subjected an epinephrine formulation to an autoclaving cycle and stability was determined after storage for one week at 60° C. More specifically, the epinephrine formulation (16 mcg/mL) was contained in a polymeric bag (250 mL printed Galenica bag with one port (Fill volume: 250 mL)) that was placed in a sealed aluminized polymeric bag (PAKVF4PCA; IMPAK CORPORATION). A single pinhole was introduced into the sealed aluminized polymeric bag using a hypodermic needle. The aluminized polymeric bag further contained an oxygen scavenger, and three different oxygen scavengers were individually tested (GLS-200, Mitsubishi Chemicals, for a non-metal-based oxygen scavenger, and SS-200 and ZPT-200 for metal-based oxygen scavengers). Autoclaving of the so packaged epinephrine solution was performed at an average temperature of 121° C. with a liquid heat-up time of 18 minutes, a purge time of 2 minutes, a liquid exposure time of 15 minutes at an average temperature of 121° C., and an exhaust time to atmospheric pressure of 36 minutes.

Results for the short-term study are shown in the Table B in which compounds eluting at RRT 1.2 and 1.3 were epinephrine formaldehyde adducts. As is readily apparent, non-metal oxygen scavengers significantly promoted adduct formation, while metal-based oxygen scavengers lead to substantially reduced adduct levels.

TABLE B
Epinephrine (16 mcg/mL)

| | | TIME POINT | | | |
|---|---|---|---|---|---|
| | | | 60° C. | | |
| | | | (1 Week) | | |
| | | Initial | GLS-200MBC | SS-200 | ZPT-200 |
| TEST | | Bulk | (non-metal-based) | (metal-based) | (metal-based) |
| Appearance | | CCS | CCS | CCS | CCS |
| pH | | 4.05 | 3.97 | 3.98 | 4.01 |
| DO (ppm) | | 6.24 | 5.18 | 5.86 | 2.25 |
| Assay | | 98.89 | 94.07 | 98.70 | 98.79 |
| Related | ADR | 0.1573 | 0.180 | 0.248 | ND |
| Substances | (Adrenochrome) | | | | |
| | Impurity C | NR | NR | NR | NR |
| | RRT- 1.080 | NR | NR | ND | ND |
| | RRT - 1.2 | 0.056 | 1.394 | 0.149 | 0.11 |
| | RRT - 1.3 | 0.057 | 1.496 | 0.146 | 0.156 |
| | RRT- 1.40 | ND | 0.064 | ND | ND |
| | RRT - 1.49 | ND | NR | ND | ND |
| | RRT - 2.365 | ND | 0.072 | ND | ND |
| | RRT- 3.743 | ND | 0.195 | 0.217 | 0.128 |
| | RRT-3.85 | 0.058 | 0.091 | 0.1 | 0.053 |
| | RRT-3.947 | 0.127 | 0.122 | 0.1312 | 0.0928 |

| | | | | |
|---|---|---|---|---|
| Total Impurities | 0.4553 | 3.614 | 0.9912 | 0.5398 |

Pinhole Study II (long-term): The inventors then subjected an epinephrine formulation to an autoclaving cycle and stability was determined after storage for one week at 60° C. More specifically, the epinephrine formulation (8 mcg/mL) was contained in a polymeric bag (250 mL printed Galenica bag with one port (Fill volume: 250 mL)) that was placed in a sealed aluminized polymeric bag (PAKVF4PCA; IMPAK CORPORATION). A single pinhole was introduced into the sealed aluminized polymeric bag using a hypodermic needle. The aluminized polymeric bag further contained an oxygen scavenger, and six different oxygen scavengers (GLS-50, Mitsubishi Chemicals, GLS-100, Mitsubishi Chemicals, GLS-200, Mitsubishi Chemicals for non-metal-based scavengers; and ZPT-100MBC, StabilOx D100-H60, and SS-100MBC for metal-based scavengers) were individually tested. Autoclaving was performed at an average temperature of 121° C. with a liquid heat-up time of 18 minutes, a purge time of 2 minutes, a liquid exposure time of 15 minutes at an average temperature of 121° C., and an exhaust time to atmospheric pressure of 36 minutes.

Results for each of the studies using the specified oxygen scavengers are shown in the tables of FIGS. 2-7 in which once more compounds eluting at RRT 1.2 and 1.3 were epinephrine formaldehyde adducts. As can be taken from Table C, a non-metal-based oxygen scavenger (GLS-50MBC) was used and chemical analysis was performed at T₀, after 1 month at 40° C., and after 2.5 months at 25° C. and at 40° C. Similarly, exemplary results are shown in Table D where a non-metal-based oxygen scavenger (GLS-100MBC) was used. Chemical analysis was performed at T₀, after 1 month at 40° C., and after 2.5 months at 25° C. and at 40° C. In yet a further test, exemplary results are shown in Table E where a non-metal-based oxygen scavenger (GLS-200MBC) was used. Once more, chemical analysis was performed at T₀, after 1 month at 40° C., and after 2.5 months at 25° C. and at 40° C.

TABLE C
Epinephrine Solution, 8 mcg/mL with GLS-50MBC (non-metal-based)

| | | TIME POINT | | | | | |
|---|---|---|---|---|---|---|---|
| | | | | | | 2.5M | |
| | | Initial | | 40° C. (1M) | | 25° C. | 40° C. |
| | | +(Vac.) | +(Vac.) | +(Vac.) | +(Vac) | +(Vac.) | +(Vac.) |
| TEST | | Bag 2 | Bag 1 | Bag 1 | Bag 2 | Bag 1 | Bag 2 |
| Appearance | | CCS | CCS | CCS | CCS | CCS | CCS |
| pH | | 3.94 | 3.94 | 3.97 | 3.98 | 3.99 | 4.0 |
| DO (ppm) | | 1.26 | 1.26 | 1.12 | 1.04 | 1.28 | 1.02 |
| Assay | | 99.009 | 99.290 | 98.9937 | 99.0872 | 99.1682 | 99.2413 |
| Related | Impurity C (Adrenalone) | 0.011 | ND | 0.0176 | 0.0196 | 0.0141 | 0.0138 |
| Substances | ADR (Adrenochrome) | ND | ND | ND | ND | ND | ND |
| | RRT - 1.2 | 0.098 | 0.101 | 0.2219 | 0.2217 | 0.2098 | 0.2468 |
| | RRT - 1.3 | 0.158 | 0.169 | 0.2238 | 0.2279 | 0.1939 | 0.1654 |
| | RRT - 3.23 | 0.025 | 0.029 | ND | ND | ND | ND |
| | RRT- 3.64 | ND | ND | ND | ND | 0.0169 | ND |
| | RRT 3.80-3.82/3.96 | 0.133 | 0.076 | ND | ND | 0.0393 | 0.0339 |
| | RRT- 4.58 | ND | ND | ND | 0.0905 | ND | ND |

| | | | | | | |
|---|---|---|---|---|---|---|
| Total Impurities | 0.425 | 0.375 | 0.463 | 0.559 | 0.474 | 0.460 |
| S-Form | 3.189 | 3.405 | NT | NT | NT | NT |
| Formaldehyde content (%) | ND | ND | ND | ND | ND | ND |
| Acetaldehyde content (%) | 0.19 | 0.19 | 0.1982 | 0.264 | ND | ND |

TABLE D
Epinephrine Solution, 8 mcg/mL with GLS-100MBC (non-metal-based)

| | | TIME POINT | | | | | |
|---|---|---|---|---|---|---|---|
| | | | | | | 2.5M | |
| | | Initial | | 40° C. (1M) | | 25° C. | 40° C. |
| | | +(Vac.) | +(vac.) | +(Vac.) | +(Vac.) | +(Vac.) | +(Vac.) |
| TEST | | Bag 2 | Bag 1 | Bag 1 | Bag 2 | Bag 1 | Bag 2 |
| Appearance | | CCS | CCS | CCS | CCS | CCS | CCS |
| pH | | 3.96 | 3.94 | 3.95 | 4.03 | 4 | 4.02 |
| DO (ppm) | | 2.67 | 2.84 | 1.17 | 1.62 | 1.21 | 1.25 |
| Assay | | 100.612 | 100.677 | 100.7811 | 100.9371 | 100.8831 | 100.8098 |
| Related | Impurity C (Adrenalone) | 0.009 | 0.009 | 0.0069 | 0.0077 | ND | ND |
| Substances | RRT- 0.76 | ND | ND | 0.0341 | ND | ND | ND |
| | RRT - 1.2 | 0.079 | 0.068 | 0.1936 | 0.1796 | 0.1571 | 0.2231 |
| | RRT - 1.3 | 0.122 | 0.078 | 0.1107 | 0.1116 | 0.1418 | 0.1863 |
| | RRT - 3.23 | 0.013 | 0.009 | ND | ND | ND | ND |
| | RRT- 3.6 | ND | ND | ND | ND | 0.0187 | ND |
| | RRT - 3.80-3.82/3.96 | 0.113 | 0.087 | 0.0284 | 0.0435 | 0.0891 | 0.0991 |
| | RRT-4.58 | ND | ND | 0.0713 | 0.0464 | ND | ND |

| | | | | | | |
|---|---|---|---|---|---|---|
| Total Impurities | 0.336 | 0.251 | 0.445 | 0.3888 | 0.4067 | 0.5085 |
| S-Form | 1.999 | 1.170 | NT | NT | NT | NT |
| Formaldehyde content (%) | ND | ND | ND | ND | ND | ND |
| Acetaldehyde content (%) | 0.19 | 0.24 | 0.3179 | 0.2715 | ND | ND |

TABLE E
Epinephrine Solution, 8 mcg/mL with GLS-200MBC (non-metal-based)

| | | TIME POINT | | | | | |
|---|---|---|---|---|---|---|---|
| | | | | | | 2.5M | |
| | | Initial | | 40° C. (1M) | | 25° C. | 40° C. |
| | | +(Vac.) | +(Vac.) | +(Vac.) | +(Vac.) | +(Vac.) | +(Vac.) |
| TEST | | Bag 2 | Bag 1 | Bag 1 | Bag 2 | Bag 1 | Bag 2 |
| Appearance | | CCS | CCS | CCS | CCS | CCS | CCS |
| pH | | 3.95 | 4.01 | 4.02 | 4.06 | 4.07 | 4.07 |
| DO (ppm) | | 1.0 | 1.13 | 1.03 | 1.32 | 1.3 | 1.09 |
| Assay | | 97.645 | 98.053 | 97.7765 | 98.0262 | 98.2175 | 98.1914 |
| Related | Impurity C (Adrenalone) | 0.005 | 0.006 | 0.011 | 0.010 | ND | ND |
| Substances | ADR (Adrenochrome) | 0.027 | ND | ND | ND | ND | ND |
| | RRT - 1.2 | 0.092 | 0.081 | 0.1513 | 0.165 | 0.1517 | 0.1951 |
| | RRT - 1.3 | 0.135 | 0.103 | 0.1716 | 0.196 | 0.1363 | 0.1827 |
| | RRT - 3.23/3.361 | 0.014 | ND | ND | ND | ND | 0.0211 |
| | RRT - 3.80-3.81/3.96 | 0.178 | 0.144 | 0.0525 | 0.1109 | 0.1333 | 0.0809 |
| | RRT- 4.58 | ND | ND | ND | 0.0423 | ND | ND |

| | | | | | | |
|---|---|---|---|---|---|---|
| Total Impurities | 0.451 | 0.334 | 0.3864 | 0.5242 | 0.4213 | 0.4798 |
| S-Form | 2.243 | 1.697 | NT | NT | NT | NT |
| Formaldehyde content (%) | ND | ND | ND | ND | ND | ND |
| Acetaldehyde content (%) | 0.28 | 0.24 | 0.3124 | 0.2901 | ND | ND |

Metal-based oxygen scavengers were tested in a similar manner to that described above for epinephrine solutions containing epinephrine at 8 mcg/mL. As can taken from Table F, a metal-based oxygen scavenger (ZPT-100MBC) was used and chemical analysis was performed at T₀, after 1 month at 40° C., and after 2.5 months at 25° C. and at 40° C. Similarly, exemplary results are shown in Table G where a metal-based oxygen scavenger (StabilOx D100-H60) was used. Chemical analysis was performed at T₀, after 1 month at 40° C., and after 2.5 months at 25° C. and at 40° C. Likewise, exemplary results are shown in Table H where a metal-based oxygen scavenger (SS-100MBC) was used. Once more, chemical analysis was performed at T₀, after 1 month at 40° C., and after 2.5 months at 25° C. and at 40° C.

TABLE F
Epinephrine Solution, 8 mcg/mL with ZPT-100 MBC (metal-based)

| | | TIME POINT | | | | | |
|---|---|---|---|---|---|---|---|
| | | | | | | 2.5M | |
| | | Initial | | 40° C. (1M) | | 25° C. | 40° C. |
| | | +(Vac.) | +(Vac.) | +(Vac.) | +(Vac.) | +(Vac.) | +(Vac.) |
| TEST | | Bag 2 | Bag 1 | Bag 1 | Bag 2 | Bag 1 | Bag 2 |
| Appearance | | CCS | CCS | CCS | CCS | CCS | CCS |
| pH | | 3.97 | 3.96 | 4.04 | 4.06 | 3.92 | 4.05 |
| DO (ppm) | | 1.37 | 2.46 | 1.21 | 1.43 | 0.72 | 1.3 |
| Assay | | 99.454 | 99.359 | 99.7633 | 99.7515 | 99.7774 | 99.7707 |
| Related | Impurity C (Adrenalone) | ND | ND | 0.0042 | 0.0063 | ND | ND |
| Substances | ADR (Adrenochrome) | ND | ND | ND | ND | ND | ND |
| | RRT - 1.2 | 0.074 | 0.067 | 0.1453 | 0.1643 | 0.1418 | 0.1681 |
| | RRT - 1.3 | 0.116 | 0.088 | 0.1268 | 0.1749 | 0.1584 | 0.1567 |
| | RRT - 3.23- | 0.015 | 0.013 | ND | ND | 0.0199 | ND |
| | RRT-3.6 | ND | ND | ND | ND | 0.0202 | ND |
| | RRT - 3.80-3.82/3.96 | 0.088 | 0.158 | 0.1027 | 0.1375 | 0.1518 | 0.1006 |
| | RRT - 4.58 | ND | ND | 0.0588 | 0.0324 | ND | ND |

| | | | | | | |
|---|---|---|---|---|---|---|
| Total Impurities | 0.293 | 0.326 | 0.4378 | 0.5154 | 0.4921 | 0.4254 |
| S-Form | 2.0313 | 1.394 | NT | NT | NT | NT |
| Formaldehyde Content (%) | ND | ND | ND | ND | ND | ND |
| Acetaldehyde Content (%) | 0.14 | 0.15 | 0.1993 | 0.1842 | ND | ND |

TABLE G
Epinephrine Solution, 8 mcg/mL with StabilOx D100-H60 (metal-based)

| | | TIME POINT | | | | | |
|---|---|---|---|---|---|---|---|
| | | | | | | 2.5M | |
| | | Initial | | 40° C. (1M) | | 25° C. | 40° C. |
| | | +(Vac.) | +(Vac.) | +(Vac.) | +(Vac.) | +(Vac.) | +(Vac.) |
| TEST | | Bag 2 | Bag 1 | Bag 1 | Bag 2 | Bag 1 | Bag 2 |
| Appearance | | CCS | CCS | CCS | CCS | CCS | CCS |
| pH | | 3.83 | 3.86 | 3.91 | 3.92 | 3.96 | 3.94 |
| DO (ppm) | | 2.73 | 4.09 | 1.05 | 0.9 | 0.76 | 0.7 |
| Assay | | 98.8852 | 98.8917 | 99.34 | 98.89 | 99.0802 | 99.5455 |
| Related | Impurity C (Adrenalone) | ND | ND | 0.0119 | 0.0142 | ND | 0.0099 |
| Substances | ADR (Adrenochrome) | ND | ND | ND | ND | ND | ND |
| | RRT- 0.583 | ND | ND | ND | ND | ND | 0.1276(Contamination, |
| | | | | | | | not real degradant peak) |
| | RRT - 1.2 | 0.0832 | 0.0856 | 0.2037 | 0.1893 | 0.1788 | 0.2386 |
| | RRT - 1.3 | 0.139 | 0.1377 | 0.1893 | 0.1637 | 0.1496 | 0.1973 |
| | RRT - 2.521 | ND | ND | ND | ND | ND | 0.0164 |
| | RRT- 2.69 | ND | ND | ND | ND | ND | 1.0045(Contamination, |
| | | | | | | | not real degradant peak) |
| | RRT - 3.23 | 0.0272 | 0.024 | ND | ND | 0.0218 | 0.0298 |
| | RRT-3.6 | ND | ND | ND | ND | 0.0227 | ND |
| | RRT - 3.80-3.82/3.96 | 0.1841 | 0.1615 | ND | ND | 0.0186 | 0.0237 |

| | | | | | | |
|---|---|---|---|---|---|---|
| Total Impurities | 0.4335 | 0.4088 | 0.4049 | 0.3672 | 0.3915 | 0.5157 |
| S-Form | 2.8625 | 2.8402 | NT | NT | NT | NT |
| Formaldehyde content (%) | ND | ND | ND | ND | ND | ND |
| Acetaldehyde content (%) | 0.22 | 0.21 | 0.4562 | 0.4713 | ND | ND |

TABLE H
Epinephrine Solution, 8 mcg/mL with SS-100 MBC (metal-based)

| | | TIME POINT | | | | | |
|---|---|---|---|---|---|---|---|
| | | | | | | 2.5M | |
| | | Initial | | 40° C. (1M) | | 25 C. | 40 C. |
| | | +(Vac.) | +(Vac.) | +(Vac.) | +(Vac.) | +(Vac.) | +(Vac.) |
| TEST | | Bag 1 | Bag 2 | Bag 1 | Bag 2 | Bag 1 | Bag 2 |
| Appearance | | CCS | CCS | CCS | CCS | CCS | CCS |
| pH | | 3.97 | 3.94 | 4.01 | 4.04 | 4.01 | 4.06 |
| DO (ppm) | | 4.35 | 3.79 | 1.23 | 1.08 | 0.76 | 0.93 |
| Assay | | 101.73 | 101.68 | 101.8 | 101.6 | 101.823 | 101.8726 |
| Related | Impurity C (Adrenalone) | 0.009 | 0.0066 | 0.0107 | 0.0106 | ND | ND |
| Substances | ADR (Adrenochrome) | ND | ND | ND | ND | ND | ND |
| | RRT - 1.2 | 0.055 | 0.075 | 0.1563 | 0.1741 | 0.1633 | 0.1935 |
| | RRT - 1.3 | 0.0585 | 0.115 | 0.1455 | 0.2166 | 0.1955 | 0.2044 |
| | RRT - 3.23 | 0.013 | 0.014 | ND | ND | ND | ND |
| | RRT - 3.361 | ND | ND | ND | ND | 0.0202 | ND |
| | RRT - 3.80-3.82/3.96 | 0.102 | 0.114 | 0.0789 | 0.1089 | 0.1169 | 0.0647 |
| | RRT-3.6 | ND | ND | ND | ND | 0.0379 | ND |

| | | | | | | |
|---|---|---|---|---|---|---|
| Total Impurities | 0.2375 | 0.3246 | 0.3914 | 0.5102 | 0.5338 | 0.4626 |
| S-Form | 1.2813 | 1.8788 | NT | NT | NT | NT |
| Formaldehyde content (%) | ND | ND | ND | ND | ND | ND |
| Acetaldehyde content (%) | 0.16 | 0.17 | 0.2198 | 0.2364 | ND | ND |

Table I provides experimental data for a control where no oxygen scavenger was used with an 8 mcg/mL solution of epinephrine. As before, chemical analysis was performed at T₀, after 1 month at 40° C., and after 2.5 months at 25° C. and at 40° C. As a further control, the inventors examined stability of the epinephrine formulations in a glass flask over time at various storage conditions and selected results are shown in Tables J and K. Autoclaving was performed at an average temperature of 121° C. with a liquid heat-up time of 2 minutes, a purge time of 2 minutes, a liquid exposure time of 15 minutes at an average temperature of 121° C., and an exhaust time to atmospheric pressure of 36 minutes. As can be clearly seen, impurities at RRT 1.2 and 1.3 are in a substantially similar range as those observed for the experiments where the metal-based oxygen scavengers were used.

TABLE I
Epinephrine Solution, No Scavenger

| | | TIME POINT | | | | |
|---|---|---|---|---|---|---|
| | | Initial | | 40° C. (1M) | | 25° C. (2.5M) |
| | | +(Vac.) | +(Vac.) | +(Vac.) | +(Vac.) | +(Vac.) |
| TEST | | Bag 1 | Bag 2 | Bag 1 | Bag 2 | Bag 1 |
| Appearance | | CCS | CCS | CCS | CCS | CCS |
| pH | | 4.03 | 3.97 | 4.09 | 4.04 | 4.07 |
| DO (ppm) | | 8.43 | 7.96 | 6.49 | 7.1 | 7.53 |
| Assay | | 100.0698 | 100.1453 | 98.2661 | 98.7358 | 99.6957 |
| Related | Impurity C (Adrenalone) | ND | ND | 0.0100 | 0.0082 | 0.0053 |
| Substances | ADR (Adrenochrome) | 0.1815 | 0.0473 | 0.1776 | 0.1060 | 0.1239 |
| | RRT - 1.2 | 0.0884 | 0.1071 | 0.2064 | 0.2175 | 0.1870 |
| | RRT - 1.3 | 0.1145 | 0.1520 | 0.2499 | 0.2158 | 0.1523 |
| | RRT - 3.23-3.30 | 0.0121 | 0.0155 | 0.0211 | 0.0270 | ND |
| | RRT - 3.64 | ND | ND | 0.0138 | 0.0178 | ND |
| | RRT-3.81/3.96 | ND | ND | 0.0128 | ND | 0.0164 |
| | RRT-4.78 | ND | ND | ND | ND | 0.0294 |
| | RRT- 3.51 | ND | ND | ND | ND | 0.0163 |
| | RRT- 4.33 | ND | ND | 0.4340 | 0.4529 | ND |
| | RRT - 4.58 | ND | ND | ND | 0.0424 | ND |
| | RRT-4.59 | ND | ND | 0.1830 | 0.2037 | ND |

| | | | | | |
|---|---|---|---|---|---|
| Total Impurities | 0.3965 | 0.3219 | 1.3086 | 1.2913 | 0.5306 |
| S-Form | 1.2176 | 2.2092 | NT | NT | NT |
| Formaldehyde content (%) | ND | ND | ND | ND | ND |
| Acetaldehyde content (%) | 0.14 | 0.17 | 0.1919 | 0.1796 | ND |

TABLE J
Epinephrine Solution, 8 μg/mL (control in flask)

| | | Epinephrine Solution, 8 μg/m L (control in flask) | | | | | | | |
|---|---|---|---|---|---|---|---|---|---|
| | | | | | | | | 1 Week | |
| | | Initial | | | | | | 2 Week | |
| | | 60° C. | | | | 3 days | | 60° C. | |
| | | Autoclaved | | Not Autoclaved | | 80° C. | | 60° C. | |
| | | Flask 1 | Flask 2 | Flask 1 | Flask 2 | Flask 1 | Flask 2 | Flask 1 | Flask 2 |
| Appearance | | CCS | CCS | CCS | CCS | CCS | CCS | CCS | CCS |
| Assay (%) | | 99.9715 | 99.9217 | 100.0324 | 99.9297 | 54.8809 | 57.9531 | 93.7071 | 95.598 |
| Dissolved Oxygen | | N/A | N/A | N/A | N/A | 8.17 | 4.89 | 8.28 | 7.9 |
| pH | | 4.02 | 4.01 | 4.04 | 3.96 | N/A | N/A | 4.17 | 4.09 |
| Related | ADR | ND | ND | 0.2262 | 0.2463 | 0.7623 | 0.6058 | 0.8716 | 0.2979 |
| Compounds* | Impurity C | ND | ND | ND | ND | 0.2948 | 0.3377 | 0.0673 | 0.0327 |
| | RRT-0.61 | ND | ND | ND | ND | 0.2866 | 0.2421 | ND | ND |
| | RRT-0.64 | ND | ND | ND | ND | ND | ND | 0.0711 | ND |
| | RRT-1.09 | ND | ND | ND | ND | ND | ND | ND | ND |
| | RRT- 0.78 | ND | ND | ND | ND | 0.0439 | ND | ND | ND |
| | RRT- 0.90 | ND | ND | ND | ND | 0.1725 | 0.118 | 0.0452 | ND |
| | RRT-1.14 | ND | ND | ND | ND | ND | ND | ND | ND |
| | RRT-1.16 | ND | ND | ND | ND | 0.2866 | 0.3495 | 0.0591 | 0.0198 |
| | RRT- 1.20 | 0.042 | 0.0469 | 0.0257 | ND | 0.9221 | 0.7132 | 0.243 | 0.1315 |
| | RRT - 1.30 | 0.0529 | 0.0507 | ND | ND | 1.654 | 1.2063 | 0.2433 | 0.134 |
| | RRT-1.65 | ND | ND | ND | ND | ND | ND | ND | ND |
| | RRT- 1.89 | ND | ND | ND | ND | 0.0714 | 0.0609 | ND | ND |
| | RRT-2.22 | ND | ND | ND | ND | 0.0854 | ND | ND | ND |
| | RRT-2.69 | ND | ND | ND | ND | 0.0194 | ND | ND | ND |
| | RRT- 2.82 | ND | ND | ND | ND | 0.142 | 0.1375 | ND | ND |
| | RRT- 2.97 | ND | ND | ND | ND | 0.1511 | 0.1583 | ND | ND |
| | RRT- 3.11 | ND | ND | ND | ND | ND | 0.0178 | ND | ND |
| | RRT-3.15 | ND | ND | ND | ND | 0.0989 | 0.0974 | ND | ND |
| | RRT-3.39 | ND | ND | ND | ND | 0.0639 | 0.0576 | ND | ND |
| | RRT- 3.361 | ND | ND | ND | ND | ND | ND | 0.0209 | ND |
| | RRT-3.355 | ND | ND | ND | ND | ND | ND | ND | 0.0163 |
| | RRT-3.402 | ND | ND | ND | ND | ND | ND | ND | 0.0162 |
| | RRT-3.44 | ND | ND | ND | ND | 0.0461 | 0.0651 | ND | ND |
| | RRT-3.47 | ND | ND | ND | ND | 0.0498 | 0.0379 | ND | ND |
| | RRT-3.51 | ND | ND | ND | ND | 0.2887 | 0.3472 | ND | ND |
| | RRT-3.57 | ND | ND | ND | ND | 0.2363 | 0.175 | 0.069 | 0.0377 |
| | RRT- 3.66 | ND | ND | ND | ND | 0.0771 | 0.0453 | ND | ND |
| | RRT- 3.70 | ND | ND | ND | ND | 0.895 | 0.8613 | 0.069 | ND |
| | RRT- 3.75 | ND | ND | ND | ND | 0.1614 | 0.1518 | ND | 0.0849 |
| | RRT- 3.80 | ND | ND | ND | ND | 0.0621 | ND | ND | ND |
| | RRT- 3.82 | ND | ND | ND | ND | 0.0565 | 0.0329 | 0.0513 | 0.0446 |
| | RRT-3.87 | ND | ND | ND | ND | 0.0329 | 0.0293 | ND | ND |
| | RRT- 2.91 | ND | ND | ND | ND | ND | ND | ND | ND |
| | RRT-3.9 | 0.2218 | 0.2507 | ND | ND | 0.208 | 0.1688 | 0.0866 | 0.0842 |
| | RRT-4.05 | ND | ND | ND | ND | 0.0344 | 0.0247 | ND | ND |
| | RRT- 4.09 | ND | ND | ND | ND | 0.1996 | 0.2003 | 0.0876 | 0.1075 |
| | RRT-4.1 | ND | ND | ND | ND | 0.0721 | 0.0547 | ND | ND |
| | RRT-4.29 | ND | ND | ND | ND | 0.2892 | 0.2861 | ND | ND |
| | RRT-4.67 | ND | ND | ND | ND | 0.0742 | 0.0943 | ND | ND |
| | RRT - 4.55 | ND | ND | ND | ND | 2.8787 | 3.0499 | ND | ND |
| | RRT-4.70 | ND | ND | ND | ND | 0.0972 | 0.1007 | ND | ND |
| | RRT-4.78 | ND | ND | ND | ND | 0.0657 | 0.0324 | ND | 0.0208 |
| | RRT-4.84 | ND | ND | ND | ND | 0.1311 | 0.1567 | ND | ND |
| | RRT-4.86 | ND | ND | ND | ND | ND | ND | 0.1456 | 0.1177 |
| | RRT- 4.93 | ND | ND | ND | ND | 0.0775 | ND | ND | ND |
| | RRT- 5.08 | ND | ND | ND | ND | 11.6497 | 13.8097 | ND | ND |
| | Total | 0.3167 | 0.3485 | 0.2519 | 0.2463 | 22.7382 | 23.8385 | 2.1306 | 1.1458 |

| | | | | | | | | |
|---|---|---|---|---|---|---|---|---|
| S-isomer | 3.5217 | 3.5518 | 0.1838 | 0.2464 | NT | NT | NT | NT |

TABLE K
Epinephrine Solution, 8 ug/mL (control in flask) (Table J Continued)

| | | Initial | | | | | | | |
|---|---|---|---|---|---|---|---|---|---|
| | | 60° C. | | | | 2 Week | | 1 Month | |
| | | Autoclaved | | Not Autoclaved | | 60° C | | 40° C. | |
| | | Flask 1 | Flask 2 | Flask 1 | Flask 2 | Flask 1 | Flask 2 | Flask 1 | Flask 2 |
| Appearance | | CCS | CCS | CCS | CCS | CCS | CCS | | |
| Assay (%) | | 99.9715 | 99.9217 | 100.0324 | 99.9297 | 84.0034 | 82.8015 | 95.678 | 97.6262 |
| Dissolved Oxygen | | N/A | N/A | N/A | N/A | 7.16 | 6.2 | 7.14 | 6.96 |
| pH | | 4.02 | 4.01 | 4.04 | 3.96 | 4.22 | 4.21 | 3.96 | 4.01 |
| Related | ADR | ND | ND | 0.2262 | 0.2463 | 0.7218 | 1.1178 | 0.4532 | 0.2115 |
| Compounds* | Impurity C | ND | ND | ND | ND | 0.1587 | 0.1404 | 0.0673 | 0.0169 |
| | RRT-0.61 | ND | ND | ND | ND | ND | ND | ND | ND |
| | RRT-0.64 | ND | ND | ND | ND | 0.0750 | 0.0909 | ND | ND |
| | RRT-1.09 | ND | ND | ND | ND | 0.0291 | 0.6376 | ND | ND |
| | RRT- 0.78 | ND | ND | ND | ND | ND | 0.0672 | ND | ND |
| | RRT- 0.90 | ND | ND | ND | ND | 0.0271 | 0.0261 | ND | ND |
| | RRT-1.14 | ND | ND | ND | ND | 0.1735 | 0.1435 | 0.0316 | ND |
| | RRT-1.16 | ND | ND | ND | ND | ND | ND | ND | ND |
| | RRT- 1.20 | 0.042 | 0.0469 | 0.0257 | ND | 0.2763 | 0.2740 | 0.16 | 0.2353 |
| | RRT - 1.30 | 0.0529 | 0.0507 | ND | ND | 0.3176 | 0.3277 | 0.1118 | 0.1069 |
| | RRT-1.65 | ND | ND | ND | ND | ND | 0.0759 | ND | ND |
| | RRT- 1.89 | ND | ND | ND | ND | ND | ND | ND | ND |
| | RRT-2.22 | ND | ND | ND | ND | ND | ND | ND | ND |
| | RRT-2.69 | ND | ND | ND | ND | ND | ND | ND | ND |
| | RRT- 2.82 | ND | ND | ND | ND | ND | ND | ND | ND |
| | RRT- 2.97 | ND | ND | ND | ND | ND | ND | ND | ND |
| | RRT- 3.11 | ND | ND | ND | ND | ND | ND | ND | ND |
| | RRT-3.15 | ND | ND | ND | ND | 0.0610 | 0.0778 | ND | ND |
| | RRT-3.39 | ND | ND | ND | ND | ND | ND | 0.0933 | 0.0701 |
| | RRT- 3.361 | ND | ND | ND | ND | ND | ND | ND | ND |
| | RRT-3.355 | ND | ND | ND | ND | ND | ND | ND | ND |
| | RRT-3.402 | ND | ND | ND | ND | ND | ND | ND | ND |
| | RRT-3.44 | ND | ND | ND | ND | 0.0799 | 0.0601 | ND | ND |
| | RRT-3.47 | ND | ND | ND | ND | ND | ND | ND | ND |
| | RRT-3.51 | ND | ND | ND | ND | 0.0588 | 0.0825 | ND | ND |
| | RRT-3.57 | ND | ND | ND | ND | 0.1712 | 0.1659 | ND | ND |
| | RRT- 3.66 | ND | ND | ND | ND | 0.0260 | 0.0357 | ND | ND |
| | RRT- 3.70 | ND | ND | ND | ND | ND | ND | ND | ND |
| | RRT- 3.75 | ND | ND | ND | ND | ND | ND | 0.099 | 0.0832 |
| | RRT- 3.80 | ND | ND | ND | ND | ND | ND | ND | ND |
| | RRT- 3.82 | ND | ND | ND | ND | 0.0410 | 0.0406 | 0.0344 | 0.0262 |
| | RRT-3.87 | ND | ND | ND | ND | ND | ND | ND | ND |
| | RRT- 2.91 | ND | ND | ND | ND | 0.0539 | 0.0697 | ND | ND |
| | RRT-3.9 | 0.2218 | 0.2507 | ND | ND | 0.1737 | 0.1336 | ND | ND |
| | RRT-4.05 | ND | ND | ND | ND | ND | ND | ND | ND |
| | RRT- 4.09 | ND | ND | ND | ND | ND | ND | ND | ND |
| | RRT-4.1 | ND | ND | ND | ND | 0.0363 | ND | ND | ND |
| | RRT-4.29 | ND | ND | ND | ND | 0.0377 | 0.0406 | ND | ND |
| | RRT-4.67 | ND | ND | ND | ND | ND | ND | ND | ND |
| | RRT - 4.55 | ND | ND | ND | ND | 2.1336 | 2.4273 | 0.7386 | 0.5634 |
| | RRT-4.70 | ND | ND | ND | ND | ND | ND | ND | ND |
| | RRT-4.78 | ND | ND | ND | ND | ND | ND | 0.0368 | 0.0332 |
| | RRT-4.84 | ND | ND | ND | ND | 0.1453 | 0.1138 | 0.1781 | 0.1498 |
| | RRT-4.86 | ND | ND | ND | ND | ND | ND | ND | ND |
| | RRT- 4.93 | ND | ND | ND | ND | ND | ND | ND | ND |
| | RRT- 5.08 | ND | ND | ND | ND | ND | ND | ND | ND |
| | Total | 0.3167 | 0.3485 | 0.2519 | 0.2463 | 4.7975 | 6.1487 | 2.0041 | 1.3965 |

| | | | | | | | | |
|---|---|---|---|---|---|---|---|---|
| S-isomer | 3.5217 | 3.5518 | 0.1838 | 0.2464 | NT | NT | NT | NT |

Therefore, it should be appreciated that non-metal oxygen scavengers significantly promoted adduct formation with epinephrine in the context of a compromised overwrap where the epinephrine was contained in a polymeric bag, while metal-based oxygen scavengers lead to significantly reduced adduct levels.

In some embodiments, the numbers expressing quantities of ingredients, properties such as concentration, reaction conditions, and so forth, used to describe and claim certain embodiments of the invention are to be understood as being modified in some instances by the term “about.” Accordingly, in some embodiments, the numerical parameters set forth in the written description and attached claims are approximations that can vary depending upon the desired properties sought to be obtained by a particular embodiment. The recitation of ranges of values herein is merely intended to serve as a shorthand method of referring individually to each separate value falling within the range. Unless otherwise indicated herein, each individual value is incorporated into the specification as if it were individually recited herein.

As used herein, the term “administering” a pharmaceutical composition or drug refers to both direct and indirect administration of the pharmaceutical composition or drug, wherein direct administration of the pharmaceutical composition or drug is typically performed by a health care professional (e.g., physician, nurse, etc.), and wherein indirect administration includes a step of providing or making available the pharmaceutical composition or drug to the health care professional for direct administration (e.g., via injection, infusion, oral delivery, topical delivery, etc.). It should further be noted that the terms “prognosing” or “predicting” a condition, a susceptibility for development of a disease, or a response to an intended treatment is meant to cover the act of predicting or the prediction (but not treatment or diagnosis of) the condition, susceptibility and/or response, including the rate of progression, improvement, and/or duration of the condition in a subject.

All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the invention and does not pose a limitation on the scope of the invention otherwise claimed. No language in the specification should be construed as indicating any non-claimed element essential to the practice of the invention.

As used in the description herein and throughout the claims that follow, the meaning of “a,” “an,” and “the” includes plural reference unless the context clearly dictates otherwise. Also, as used in the description herein, the meaning of “in” includes “in” and “on” unless the context clearly dictates otherwise. As also used herein, and unless the context dictates otherwise, the term “coupled to” is intended to include both direct coupling (in which two elements that are coupled to each other contact each other) and indirect coupling (in which at least one additional element is located between the two elements). Therefore, the terms “coupled to” and “coupled with” are used synonymously.

It should be apparent to those skilled in the art that many more modifications besides those already described are possible without departing from the inventive concepts herein. The inventive subject matter, therefore, is not to be restricted except in the scope of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced. Where the specification claims refers to at least one of something selected from the group consisting of A, B, C . . . and N, the text should be interpreted as requiring only one element from the group, not A plus N, or B plus N, etc.

1. A method of protecting a catecholamine in a catecholamine-containing formulation from inadvertent formaldehyde condensation product formation, comprising:
placing a polymeric container into an overwrap, wherein the polymeric container encloses the catecholamine-containing formulation;
placing a metal-containing oxygen scavenger in a space between the polymeric container and the overwrap; and
sealing the overwrap from the environment to form a sealed overwrap;
wherein the inadvertent formaldehyde condensation product formation is triggered by improper sealing or puncture of the overwrap.

2. The method of claim 1, wherein the catecholamine comprises epinephrine, norepinephrine, isoproterenol, or combinations thereof.

3. The method of claim 1, wherein the polymeric container is formed from polyvinyl chloride, polyethylene, polypropylene, polyethylene terephthalate, an ethylene-propylene copolymer, ethylene vinyl acetate, a copolyester ether polymer, or combinations thereof.

4. The method of claim 1, wherein the polymeric bag has a volume of between 10 mL and 12,000 mL.

5. The method of claim 1, wherein the overwrap is a metalized polymer overwrap.

6. The method of claim 1, wherein the metal-containing oxygen scavenger comprises iron.

7. The method of claim 1, wherein the puncture has an area of less than 5 mm².

8. The method of claim 1, wherein less than 1% of formaldehyde adduct are formed at 25° C. within 1 month following break or puncture.

9. A kit for use in protecting a catecholamine in a catecholamine-containing formulation from inadvertent formaldehyde condensation product formation, the kit comprising:
an overwrap;
a polymeric container comprising a catecholamine, the polymeric container contained in the overwrap; and
a metal-containing oxygen scavenger in a space between the polymeric container and the overwrap;
wherein the oxygen scavenger is present in an amount that prevents inadvertent formaldehyde adduct formation upon a break or puncture of the overwrap.

10. The packaged pharmaceutical composition of claim 9, wherein the catecholamine comprises epinephrine, norepinephrine, isoproterenol, or combinations thereof.

11. The packaged pharmaceutical composition of claim 9, wherein the metal-containing oxygen scavenger comprises iron.

12. The packaged pharmaceutical composition of claim 9, wherein the oxygen scavenger is present in an amount that leads to equal or less than 1% of formaldehyde adduct at 25° C. within 1 month following the break or puncture.

13. The packaged pharmaceutical composition of claim 9, wherein the break or puncture an area of less than 5 mm².

14-20. (canceled)

21. A method of protecting a catecholamine in a catecholamine-containing formulation from formaldehyde condensation product formation, comprising:
placing a polymeric container into an overwrap, wherein the polymeric container encloses the catecholamine-containing formulation;
placing a metal-containing oxygen scavenger in a space between the polymeric container and the overwrap; and
partially, but not completely, sealing the overwrap from the environment to form a partially sealed overwrap;
wherein the formaldehyde condensation product formation is influenced by the presence of oxygen resulting from partial sealing of the overwrap.

22. The method of claim 21, wherein the catecholamine comprises epinephrine, norepinephrine, isoproterenol, or combinations thereof.

23. The method of claim 21, wherein the polymeric container is formed from polyvinyl chloride, polyethylene, polypropylene, polyethylene terephthalate, an ethylene-propylene copolymer, ethylene vinyl acetate, a copolyester ether polymer, or combinations thereof.

24. The method of claim 21, wherein the polymeric bag has a volume of between 10 mL and 12,000 mL.

25. The method of claim 21, wherein the overwrap is a metalized polymer overwrap, and/or wherein the metal-containing oxygen scavenger comprises iron.

26. (canceled)

27. The method of claim 21, wherein the partially sealed overwrap has an opening having an area of less than 5 mm².

28. The method of claim 21, wherein less than 1% of formaldehyde adduct are formed at 25° C. within 1 month following partial sealing of the overwrap.

29-33. (canceled)
